(19) United States
(12) Patent Application Publication
(10) Pub. No.: US 2003/0153812 A1
(43) Pub. Date: Aug. 14, 2003

(54) Method of examining potential cellular abnormalities

(76) Inventors: Iain Louis Hutchison, London (GB); Robert Jonathan Cull, Tewkesbury (GB)

Correspondence Address: CANTOR COLBURN, LLP, 55 GRIFFIN ROAD SOUTH, BLOOMFIELD, CT, 06002

(21) Appl. No.: 10/324,529

(22) Filed: Dec. 19, 2002

(30) Foreign Application Priority Data
Dec. 22, 2001 (GB) 0130862.6

(51) Int. Cl. A61B 1/06

(52) U.S. Cl. 600/168

(57) ABSTRACT

A probe of a video microscope system has a window sealed to a casing, the window having a flat outer surface for contacting an area of the body of a patient, such as the patient's mouth In the method of the invention, a dye is administered to the body area so as to stain both normal and any abnormal cells, which stained area is then examined with magnification means of the probe so as to allow a surgeon to interpret the images and identify any cancerous cells.

This invention relates to a method of examining potential cellular abnormalities and also to apparatus for use in said method. Particularly, but not exclusively the method is for the examination of potential mouth cancer.

The majority of malignant lesions in a patient's oral cavity are squamous cell carcinomas. A proportion of these are preceded by distinctive lesions which have pre-malignant status, The assessment of these lesions is currently made by repeated observation over a period of time and by incisional biopsy on one or several occasions.

The clinical examination of these lesions at one point in time does not provide a robust diagnosis in the majority of cases. It is the repeated observation by one observer which will in most cases lead to a diagnosis of progression to cancer. Unfortunately patients with dysplasia usually exhibit widespread change throughout the whole oral cavity, making biopsy of such lesions difficult. It is therefore possible to have a falsely reassuring histological diagnosis with frank carcinoma at a separate site. Also, patients with proven carcinoma or severe dysplasia are followed up regularly and have a 20% chance of further malignant change. This usually involves repeated incisional biopsy. Therefore, the current standard follow-up examination of patients with pre-malignant lesions, or who have had previous malignancy, is intermittent biopsy when suspicion is raised by simple naked eye observation.

It is known that alongside a soft tissue examination of the mouth, dentists now use a special mouthwash to help detect barely visible, asymptomatic lesions and pre-cancerous cells. It is a simple, painless short procedure. The screening solution mainly used at present, called OraTest, is a blue dye containing tolonium chloride. The dye, which is swilled around the mouth and then spat out by the patient, is retained on roughened patches giving them a bluish coloration, These patches are usually white or red and may be premalignant.

However, because the dye is so sensitive and can sometimes also stain what turn out to be harmless lesions, ulcers, or even skin tissue burnt by hot food, the test has to be given in two stages. If the first test shows anything suspicious, the area is photographed and the test repeated two weeks later, when a firer photograph is taken and the results compared. Harmless lesions or ulcers should have cleared up by then, but if the second test stains the original suspicious patch, the patient is immediately referred to an oral surgeon who will then take a biopsy and make a final diagnosis.

Although the described test has its advantages, it is often the case that the symptoms of mouth cancer are straightforward and easy to recognise, including ulcers which do not heal within four weeks, red or white patches in the mouth, or persistent one-sided sore throats. Consequently with the test described, the lesions stained are often ones which can be seen anyway without the necessity for this test.

However, not all patients referred to oral surgeons have an existing cancer. Some have a large pre-cancerous white patch, of which only ten per cent will transform into cancer. The difficulty facing the surgeon is how to decide which area is likely so to transform. It is clearly undesirable to remove all the lining of the mouth just to be sure. Presently, however, the practice is to carry out a biopsy on every patient.

An object of the invention is to provide an improved method of examining potential cellular abnormalities and also apparatus for carrying out the method.

According to one aspect of the invention, there is provided a probe for use in a microscope system, the probe comprising an outer casing having at least partly therein an optical lens arrangement for producing a magnified image of a surface contacted by the probe, in use, and illumination means, wherein the casing has therein a window which is sealed thereto against ingress of liquid, which window has a flat outer surface flush with or proud of the exterior surface of the casing therearound, so that, in use, said flat outer surface can provide said contact of the probe with said surface.

Preferably said window is of transparent quartz and is desirably fused to said casing, which is conveniently of stainless steel. If necessary a paste material can be provided to form said seal of the window. Desirably the probe includes means for linking the probe to a control unit of the microscope system. Conveniently the probe is connectable, in use, to cable means which itself is connectable, in use, to the control unit.

According to a further aspect of the invention, there is provided a method of examining potential cellular abnormalities comprising administering to a body area a dye which stains/appears to stain the DNA and RNA and therefore the nuclei of all cells, which are then examined with magnification means.

The nuclei of cancer cells stain/appear to stain in a distinctive and different way from normal cells and this can be seen with the magnification means,

Preferably the method is for examining lesions/pre-cancerous patches of the body area for subsequent determination, for example, whether the diagnostic methods i.e. a biopsy, need be performed thereon. Desirably vital dyes or stains such as 3,7-bis (dimethylamino)phenzathionium chloride or 3-amino-7-dimethylamino-2-methylphenazathionium chloride are used to stain the tissues and cells. Conveniently the suspiciously stained part(s) of the body area is or are examined using a microscope, with a probe of which being placed in contact with the stained part or parts to produce a magnified image thereof. Thus the method is not a diagnostic method, but merely identifies areas which should be biopsied, i.e. on which a diagnostic method should be carried out.

The invention will now be described, by way of example, with reference to the accompanying drawings, in which:

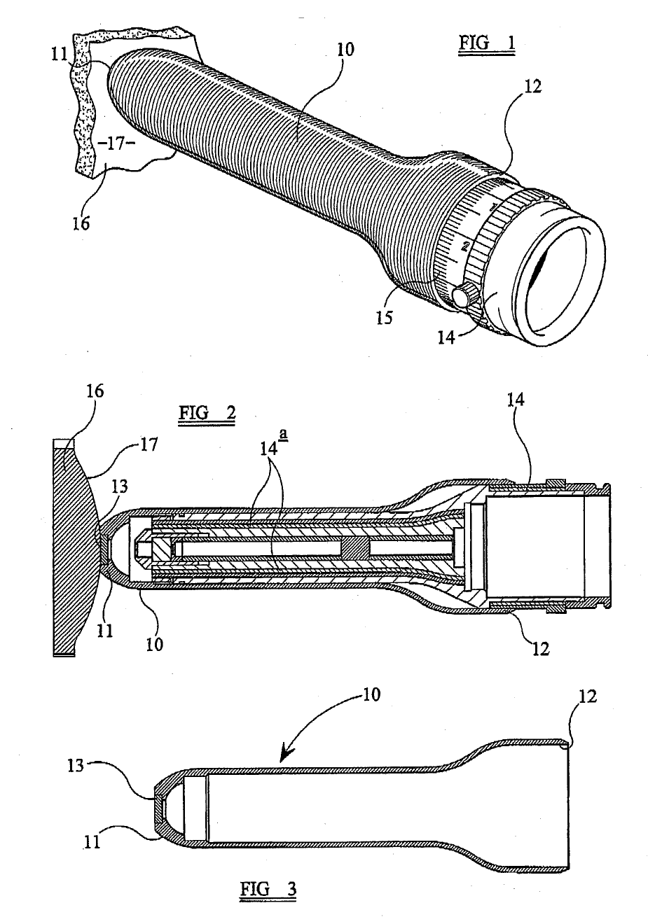

FIG. 1 is a perspective view of a probe of the invention shown in contact with an area of sin,

FIG. 2 is a longitudinal cross-sectional side view of FIG. 1,

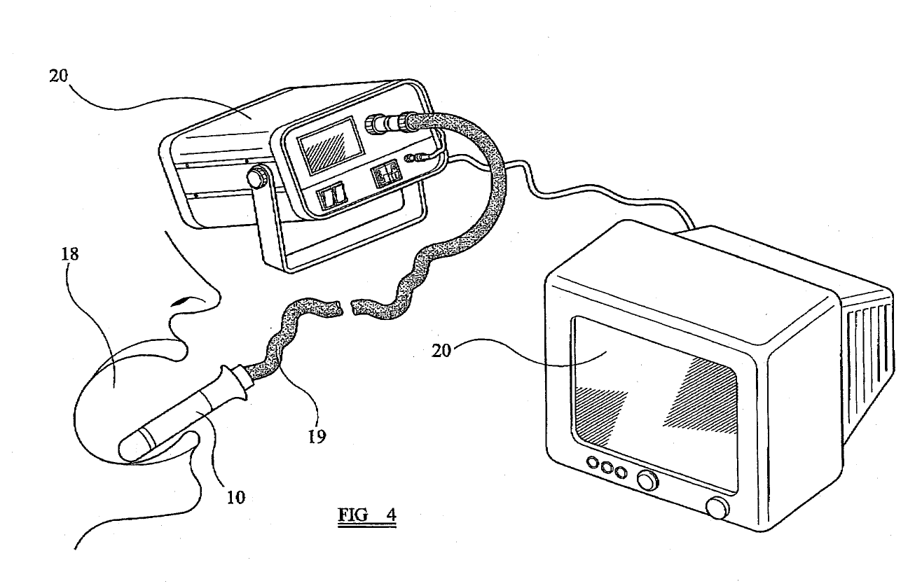

FIG. 3 is a longitudinal cross-sectional view of a casing of the probe, and

FIG. 4 schematically shows the probe used in a method of the invention, where it is connected to a control unit, which is connected to a monitor.

Although the method and apparatus to be described below has been developed, and will be described, specifically in relation to mouth cancer, it is to be noted that the method is applicable more generally, for example to examine any selected body area where lesions/pre-cancerous patches or the like have been found. In particular the method has application in examining for cervical cancer or colonic cancer, and in all cases the apparatus described hereinafter can be used.

As far as examining for mouth cancer is concerned, the invention satisfies various aims, namely:

1. To develop a method of examining the oral mucous membranes in vivo,

2. To develop a technique for assessing the pathological status of lesions in the oral cavity,

3. To assess the pre-malignant status of lesions using a contact microscope within the oral cavity and to correlate these results with clinical and histopathological assessment by a surgeon,

4. To assess the inter-observer variation with respect to diagnosis of malignancy using the contact microscope.

In general the method, in one embodiment, involves a patient using a vital dye, such as ones mentioned above, so that there is staining of the DNA and RNA, and thus the nuclei of any pre-cancerous cells. The suspiciously stained part or parts of the treated area can then examined under a special microscope in order to provide a magnified image. This enables very small cells which would otherwise not be visible to the human eye, to be examined. These images can conveniently be viewed on a video monitor. As a consequence this allows an oral surgeon to interpret these enlarged images to the extent that the surgeon can identify any cells which it is believed will transform into cancer, at the same time allowing the surgeon to identify any benign areas, thereby allowing the patient to be reassured.

Vital dyes or stains are dyes which stain living tissues as opposed to those dyes that only stain tissue that has been previously fixed (and has therefore been removed from the body), Reference is not made to any particular chemical composition, but just to this property of the dyes or stains. Examples include:

Rhodamine; and Ethidium bromide; and possibly

Rose bengal

Fluorescein

Indian ink

Lissaimine green B

Methylene blue=methyl thioninium chloride or

3,7-bis(dimethylamino)phenzathionium chloride

Toluidine blue=Tolonium chloride or

3-amino-7-dimethylamino-2-methylphenazathionium chloride

(These are both ‘Thiazine dyes’ and ‘heterocyclic compounds with one ring’).

The apparatus is conveniently a video microscope, preferably a compact video microscope, which comprises a control unit 20 with integral light source, to which is connected a video monitor 21 and a video probe head. Alteratively a personal computer (PC) may be utilised in place of the video monitor, with the images obtained by the microscope being imported onto the PC using an image capture card. In one embodiment, the microscope is digital. These images may be archived onto compact disc using propriety image manipulation software.

Although video microscopes are known, where a manually focused lens arrangement is provided at the operative end of the video probe head, such a probe would not be suitable for the method of the present invention, particularly in that it is not suitable for sterilisation. Accordingly a new form of probe has been designed for use with die, method of the invention, and this is shown in FIGS. 1 to 3. The probe is made up of a casing 10 in the form of an outer sleeve preferably of stainless steel. This casing is of generally tubular form having a rounded operative end 11 and an enlarged open opposite end 12. The smaller operative end 11 is closed by a window 13, which is circular, but could be of square or rectangular form, being sealingly fitted in a complementarily shaped central opening in said end 11 of the casing, as shown in FIGS. 2 and 3. The window 13 is preferably of transparent quartz material, and in one embodiment the quartz material is fused in place so as to provide a liquid-tight, and also preferably air-tight, seal. If necessary, a suitable paste material could be provided between the quartz and the stainless steel of the casing. By virtue of this seat, it becomes possible for sterilisation of this operative end of the probe.

Within the casing 10, as shown in FIG. 2, is an optical lens arrangement 14 which comprises any suitable form of manually focusable lens, for example a zoom lens or the like, with the manual adjustment means being provided just outside the end 12 of the casing. As shown in FIG. 1, this adjustment can incorporate a suitable scale 15 in a conventional manner. The lens arrangement features, in this embodiment, integral fibreoptic lighting 14a to illuminate the operative end 11 of the probe with the illumination passing through said window onto said object 16 being examined, for example a body area such as the inside of the mouth 18. The outer end of the lens arrangement is adapted for connecting the probe to a cable 19 or the like, which is itself connected, in use, to a control unit 20 of the video microscope. The probe could alternatively be linked to the control unit in any other suitable manner.

It is important to note, as shown in FIG. 2, that the outer flat surface of the window is flush with the exterior surface of the casing at said operative end, or that alternatively said flat outer surface of the window is proud thereof, so as to allow contact with the skin 17 of the body area being examined. This is important in that when used in the mouth 18, the contact tends to force saliva away from the skin/tissue, and with the skin becoming transparent, the cells which it is wished to view become clearly visible as the magnified image produced by the video microscope. Thus various important changes have been made to this probe as compared to the equivalent component of known video microscopes, namely the particular design and arrangement of the window, the provision of the ‘stretched’ optics of the probe and consequently the increased depth of field, in addition to the sterilisation aspect mentioned above.

Accordingly, in use, when examining lesions in a patient's mouth, the vital dye is initially administered, this being swilled around the patient's mouth and then spat out, resulting in staining of the DNA and RNA and therefore the nuclei of all cells. The suspicious area or areas is or are then examined by using the probe shown in FIGS. 1 to 3 with the exterior surface of the window being placed in contact with the suspicious area or areas at the inside of the patient's mouth. By looking at the magnified/enlarged images a surgeon is able to use his/her judgement to identify dangerous cells and biopsy these suspicious areas to confirm or refute that these are cancerous. Moreover by this method it is also possible to identify patients who are in the clear, thereby avoiding the need for a biopsy or surgery. Thus by greatly magnifying the examined cells, it is possible to overcome the problems referred to with the known method in that it is subsequently possible for the very detailed images of the cells to be interpreted expertly, whereas this is not possible with a normal visual inspection of the stained areas in a patient's mouth.

As far as the potential benefits are concerned, reference is made to the following:

1. May lead to immediate suspicion of oral carcinoma.

2. May lead to immediate suspicion of benign oral lesions, therefore reassuring patients.

3. Can allow selection of intraoral biopsy sites in the setting of widespread changes within the oral cavity.

4. Will improve regular surveillance of patients with premalignant lesions (only 10% of whom will progress to develop cancer at these areas) and possibly reduce the need for regular biopsies in these patients.

5. Can allow the ability to plan major oncological surgery without the need to wait for histological results.

6. Surveillance of post-operative patients following resection for carcinoma, avoiding the need for recurrent biopsy.

7. Can allow further research into the assessment of pre-malignancy using complementary techniques.

8. May lead to the earlier detection of cancer in some patients reducing treatment costs, treatment morbidity and improving their chances of cure.

1. A probe for use in a microscope system, the probe comprising an outer casing having at least partly therein an optical lens arrangement for producing a magnified image of a surface contacted by the probe, in use, and illumination means, wherein the casing has therein a window which is sealed against ingress of liquid, which window has a flat outer surface flush with or proud of the exterior surface of the casing therearound, so that, in use, the flat outer surface can provide contact of the probe with said surface.

2. A probe as claimed in claim 1, wherein the window is used to said casing.

3. A probe as claimed in claim 1, wherein a paste is provided between the window and the casing.

4. A probe as claimed in claim 1, wherein the window is of transparent quartz.

5. A probe as claimed in claim 1, wherein the outer casing is of steel.

6. A probe as claimed in claim 1, wherein the outer casing is generally tubular and die window is formed in a rounded, operative end thereof.

7. A method of examining potential cellular abnormalities comprising administering to a body area a dye which stains/appears to stain the DNA and RNA and therefore the nuclei of all cells, and examining the stained area with magnification means.

8. A method as claimed in claim 7, wherein the dye administered is a vital dye.

9. A method as claimed in claim 8, wherein the dye is Methylene blue.

10. A method as claimed in claim 8, wherein the dye is Toluidine blue.

11. A method as claimed in claim 7, wherein the stained area is examined using a probe of a microscope system.

12. A method as claimed in claim 11, wherein the probe has a window in a casing thereof, which window has a flat outer surface which is placed in contact with a part or parts of the stained area.

13. A method as claimed in claim 12, wherein an image of said part or parts, magnified by said magnification means, is viewed on a monitor.

14. A method as claimed in claim 11, wherein the microscope system is a digital microscope system.

15. A method as claimed in claim 7, wherein the body area is the interior of a patient's mouth.
